(19) United States
(12) Patent Application Publication
(10) Pub. No.: US 2026/0316050 A1
(43) Pub. Date: Oct. 15, 2026

(54) PROCESS FOR PREPARATION OF SUPEROLEOPHILIC ABSORBENT AND A METHOD OF ITS UTILIZATION

(71) Applicant: Indian Oil Corporation Limited, Mumbai (IN)

(72) Inventors: Pragya N/A, Faridabad (IN); Prakash Chandra Sahoo, Faridabad (IN); Manoj Kumar, Faridabad (IN); Amardeep Singh, Faridabad (IN); Ravi Prakash Gupta, Faridabad (IN); Umish Srivastva, Faridabad (IN)

(73) Assignee: Indian Oil Corporation Limited, Mumbai, MH (IN)

(21) Appl. No.: 19/336,396

(22) Filed: Sep. 22, 2025

(30) Foreign Application Priority Data
Sep. 20, 2024 (IN) 202421071270

(51) Int. Cl. B01J 20/20; B01J 20/02; B01J 20/28; B01J 20/30; B01J 20/32; C09K 3/32; C12N 1/20

(52) U.S. Cl. CPC B01J 20/205; B01J 20/0218; B01J 20/0266; B01J 20/28011; B01J 20/28045; B01J 20/3085; B01J 20/3212; B01J 20/3217; B01J 20/3223; B01J 20/3236; B01J 20/324; B01J 20/3289; C09K 3/32; C12N 1/20

(57) ABSTRACT

The present disclosure relates to a process for modification of melamine sponge and a method of its utilization thereof. The process includes contacting a three-dimensional structure of a melamine sponge with a pre-grown bacterial culture at a temperature in a range of 25° C.-40° C. for 24-72 hours to obtain an etched porous substrate. The etched porous substrate is coated with a one-dimensional metal complex, a two-dimensional metal complex, or a transition metal complex to obtain a coated etched porous substrate. The coated etched porous substrate is dip contacted with an alkylsiloxane dispersed in a first solvent for a duration between 10 seconds to 300 minutes and curing at a temperature between 60° C. and 190° C. to obtain alkylsiloxane coated structure. The alkylsiloxane coated structure is coated with an alkylsilane dispersed in a second solvent for a duration between 10 seconds to 300 minutes to obtain a modified 3D sponge. The modified 3D sponge is washed with a solvent and cured at a temperature between 60° C. and 210° C. to obtain the modified melamine sponge.

This application claims priority to Indian Application No. 202421071270 filed on Sep. 20, 2024. The contents of this application are hereby incorporated by reference in its entirety.

### FIELD OF THE INVENTION

The present invention relates to a process for preparation of super-oleophilic absorbent and a method of its utilization thereof. Specifically, the present invention relates to a process for modification of melamine sponge and a method of its utilization thereof. More specifically, the present invention relates to a simple, scalable and economical process or method to fabricate/modify melamine sponge with super-hydrophobic qualities for large-scale offshore oil spills having excellent water repellence, superior adsorption performance, outstanding reusability having ability to withstand multiple cycles of sorption-squeezing and low material cost.

### BACKGROUND OF THE INVENTION

Accidental spillage of oils and organic solvents along industrial development causes severe environmental and ecological problems. These environmental issues lead to health problems for human beings, marine mammals, aquatic ecosystems and plant. Several methods have been proposed to address the problems, including bioremediation, in situ burning, skimming, and using chemical dispersant. Bioremediation is a method that uses specific micro-organisms such as bacteria and some species of plants to clean up oil spills. In situ burning is burning floating oil at the location of the spill. The most common technique to remove the oil slicks is skimming, which used tools to clean the surface of water from oil. Using dispersants on the oil spill to break down the oil into droplets that easily mix with water. All the above methods are costly, time consuming, and low efficiency. These generate secondary pollutants having serious undesirable effects.

Cleanup of the spilled oil in water has been challenging in the huge volumes. The most effective and cost convenient way to clean and recollect oil from oil spillage is the use of sorbents. In order to separate oil from water medium, the absorbent material should be hydrophobic (water contact angle>90°) as well as oleophillic in nature having high absorption ability, recyclability, and low cost. There is plethora of literature available related to such absorbents. Conventional absorbents viz-natural wood sawdust, zeolite and wool fibres, and nonwoven polypropylene/polyester fabrics typically have the drawbacks of low absorption capacities, poor selectivity, and poor recyclability.

Recently, three-dimensional porous materials have gained attention due to their significant application in the absorption of toxicant organic and oil pollutions. The melamine sponge is a type of three-dimensional porous material that is commercially available and can be used for fabrication of oil absorbents due to its excellent absorption ability, ultra-low density, low cost, high porosity, and good compression strength.

Melamine sponge (MS) have attracted great attention for oil-water separation due to their low cost, good flexibility, three-dimensional structure, and facile design. However, MS is naturally amphipathic, which restricts the removal of oils from oil-water mixtures. In order to separate oil from water medium, it should be hydrophobic (water contact angle>90°) and oleophilic. The wettability of a solid surface is determined by its chemical composition and the surface roughness. Lowering surface energy and increasing surface roughness are two common strategies for the preparation of hydrophobic surfaces. Several reports are available for development of hydrophobic melamine sponge-like Surface Modification by Organosilicons with vinyltrimethoxysilane (VTMS), octadecylsiloxane (ODS), octadecyltrichlorosilane (OTS), potassium methyl silicate CH₃Si(OK)₃ etc; Surface Modification by Carbon-Based Materials namely graphene, CNTs etc; Surface Modification by MOFs like Cu doped ZIF-67 nanoparticles. Other materials have been also reported to modify hydrophillic three-dimensional foam to hydrophobic for efficient oil water separation-Fe₃O₄ nanoparticles, Acrylate monomers, cellulose nanocrystals (CNC) etc.

There is varied plethora of prior art available for modification of hydrophillic foam to change its wettability to a superhydrophobic surface.

There is varied plethora of literature available for surface modification of three-dimensional materials for developing hydrophobic Sponges for Oil/Water Separation. Wang et al. 2014, modified a MS by dip coating the surface with SiO₂ nanoparticles to improve surface roughness, which was silanized with vinyltrimethoxysilane (VTMS). Ke et al. 2014, modified a MS with octadecylsiloxane (ODS) and octadecyltrichlorosilane (OTS) resulting in WCA of 153±1°. Dickerson et al. (2014) immersed in a toluene solution of OTS. It then formed hydrogen bonds with the amine group of the sponge generating a self-assembled layer to be covalently bonded to the sponge skeleton making it hydrophobic. Melamine sponge was modified in another study (Wang et al. 2017) by immersion in an aqueous solution of potassium methyl silicate CH₃Si(OK)₃, which formed a threadlike organosilane layer on the sponge surface resulting in increased surface roughness.

Tai et al. (2012) modified a MS with graphene nanosheets and PDMS. Again, Tai et al. in 2018 modified MS with reduced graphene oxide sheets and a thin polydimethylsiloxane film. The modified sponge demonstrated good recyclability for 10 cycles. Hsiao et al. (2016) modified a MS with TiO2 nanoparticles, trifluoroacetic acid (TFAA), and PDMS to modify the surface. TiO2 nanoparticles functionalized with TFAA were first coated on the sponge, which made it rough with hierarchical roughness. The sponge was then immersed in a PDMS/toluene solution to improve the adhesion between the nanoparticles and the sponge. Feng et al. (2019) fabricated a superhydrophobic MS by co-depositing the sponge with polydopamine (PDA) containing Fe₃O₄ nanoparticles and PDMS.

Wang et al. (2020) modified a MS with Cu doped ZIF-67 nanoparticles and PDMS. For which the sponge was first immersed in a Cu/ZIF-67 methanol solution, and then PDMS coating was applied. The porous structure of MOF facilitates protecting low surface energy materials to achieve self-healing. Li et al. (2019) made a hydrophobic composite material by modifying a MS with carboxymethyl-cellulose sodium (CMC) and HPU-13. Jiang et al. (2019) prepared a hydrophobic MS via the self-assembly of ZIF-67 (zeolitic imidazolate framework-67) nanoparticles onto the sponge skeleton with excellent stability in harsh conditions.

A two-tier hierarchical structured superhydrophobic-superoleophilic melamine sponge was developed using polypyrrole walls and Ag nanoparticles by Chen et al. (2017). Another study reported kaolinite modified graphene oxide-melamine sponge (K-GOMS), where graphene oxide increased roughness of the sponge smooth surface and hydrophobic kaolinite adhered on it.

U.S. Ser. No. 11/214,646B2 invention discloses highly hydrophobic and oleophilic melamine resin via metal-ion induced wettability transition, application, and preparation by a one-step solution immersion process.

US20170036190A1 discloses a superhydrophobic sponge as an efficient oil absorbent material for oil spill cleanup applications. The hydrophobic or super-hydrophobic compositions include the reaction product of at least a substrate having a reacting group which reacts with a silane and an alkylsilane or a fluoroalkylsilane.

U.S. Ser. No. 10/850,258B2 directs towards a method for manufacturing a modified porous composite structure, comprising a functionalized melamine sponge modified with Coordination polymers, e.g. metal-organic frameworks [MOF], zeolitic imidazolate frameworks [ZIF].

Despite many significant progresses in preparation of hydrophobic and oleophilic materials in recent years, application of the hydrophobic materials as oil absorbents has been limited due to complicated manufacturing processes, high cost, difficulty to scale up, lower recyclability, lower absorption capacity, higher surface energy, lower stability, low availability of raw materials limiting its industrial scale utilization. For instance, fluorine modifiers are common low surface energy material, having excellent properties such as weather resistance, refractory, corrosion resistance, and oxidation stability but are expensive requiring complex preparation process.

Another practical issue with usage is its reuse and oil recovery from sponges. Lower extraction of absorbed oils greatly limited their potential for practical oil spill cleanup applications. Furthermore, almost all of the superhydrophobic fabricated sponges exhibit good oil sorption capacity and recyclability with distillation as the oil extraction method from them. But their recyclability by squeezing for recovering oil remains quite poor. This is because during the squeezing process, the hydrophobic coatings easily detach themselves from the skeletons due to the weak adhesion/bonds/forces between the coatings and base matrix. For all practical purposes squeezing and compression appears to be the only on-site, fast, and cost-effective extraction method for recovering absorbed oil.

Therefore, it is highly desirable to develop a simple, scalable and economical method to fabricate melamine sponge with super-hydrophobic qualities for large-scale offshore oil spills having excellent water repellency, superior adsorption performance, outstanding reusability having ability to withstand multiple cycles of sorption-squeezing and low material cost. The present invention aims to find solution to above limitations for possible industrial scaling up.

### OBJECTS OF THE INVENTION

The main object of the present invention is to develop a simple, scalable and economical method to fabricate melamine sponge with super-hydrophobic qualities for large-scale offshore oil spills having excellent water repellency, superior adsorption performance, outstanding reusability having ability to withstand multiple cycles of sorption-squeezing and low material cost.

Another object of the present invention is to develop an industrial scaling-up method to fabricate melamine sponge with super-hydrophobic qualities.

### SUMMARY OF THE INVENTION

The present invention discloses a process for fabrication or modification of a melamine sponge, comprising: i. contacting three-dimensional structure of a melamine sponge with pre-grown bacterial culture in range of 25-40° C. for 24-72 hours and allowing to grow the cultures in a medium to obtain an etched porous substrate; ii. coating the etched porous substrate with a one-dimensional or two-dimensional or transition metal complexes material to obtain a coated etched porous substrate; iii. dip contacting the above with an alkylsiloxanes in a solvent for a duration between 10 seconds to 300 minutes and curing at elevated temperature to obtain alkylsiloxane coated structure; iv. contacting the above alkylsiloxane coated structure with an alkylsilane in a solvent for a duration between 10 seconds to 300 minutes to obtain a modified 3D sponge; v. washing the modified 3D sponge repeatedly with a solvent and cured at elevated temperature to obtain the modified melamine sponge.

### BRIEF DESCRIPTION OF DRAWINGS/FIGURES

FIG. 1 is schematic representation of the process (four steps) of the present invention.

Step 1: Illustrates incubated melamine sponge with acidophile

Step 2: Illustrates dip coating with any two-dimensional or one-dimensional or transition metal complexes.

Step 3: Illustrates dip coating with PDMS/alkylsiloxane and followed by temperature curing.

Step 4: Illustrates dip coating with OTS/alkylsilane and temperature curing followed by solvent wash.

### DETAILED DESCRIPTION OF THE INVENTION

In an aspect of the present invention, the present invention discloses a process for fabrication or modification of a melamine sponge, comprising: i. contacting three-dimensional structure of a melamine sponge with pre-grown bacterial culture in range of 25-40° C. for 24-72 hours and allowing to grow the cultures in a medium to obtain an etched porous substrate; ii. coating the etched porous substrate with a one-dimensional or two-dimensional or transition metal complexes material to obtain a coated etched porous substrate; iii. dip contacting the above with an alkylsiloxanes in a solvent for a duration between 10 seconds to 300 minutes and curing at elevated temperature to obtain alkylsiloxane coated structure; iv. contacting the above alkylsiloxane coated structure with an alkylsilane in a solvent for a duration between 10 seconds to 300 minutes to obtain a modified 3D sponge; v. washing the modified 3D sponge repeatedly with a solvent and cured at elevated temperature to obtain the modified melamine sponge.

In an embodiment of the present invention, the pre-grown bacterial culture is selected from Acidobacteriota, Acidithiobacillales, Thiobacillus, Acetobacter, Alicyclobacillus or a combination thereof.

In an embodiment of the present invention, the medium comprising potassium dihydrogen phosphate (KH₂PO₄) in an amount of 2-5 g, magnesium sulfate heptahydrate (MgSO₄·7H₂O) in an amount of 0.25-2.0 g, ammonium sulfate [(NH₄)₂SO₄] in an amount of 2-5 g, calcium chloride dihydrate (CaCl₂·2H₂O) in an amount of 0.1-1.0 g, sodium thiosulfate pentahydrate (Na₂S₂O₃·5H₂O) in an amount of 2-8 g.

In an embodiment of the present invention, the one-dimensional or two-dimensional or transition metal complexes material is selected from reduced graphene, CNTs, or MoS₂@CNTs or MoS₂@GO or g-C3N4 (hydrophobic).

In an embodiment of the present invention, concentration of the one-dimensional or the two-dimensional or the transition metal complexes in a solvent range from about 1 ppm to 5000 ppm.

In an embodiment of the present invention, the alkylsiloxane is selected from polyvinyl siloxane, polyethermethylsiloxanes (PEMS), hexamethyldisiloxane, hexamethylcyclotrisiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, polydimethylsiloxane or a combination thereof.

In an embodiment of the present invention, concentration of the alkylsiloxane in a solvent range from about 0.05 wt. % to about 100 wt. %.

In an embodiment of the present invention, the alkylsilane is selected from octadecyltrichlorosilane, dodecyltrichlorosilane, octyltrichlorosilane, butyltrichlorosilane, hexyltrichlorosilane, or a combination thereof.

In an embodiment of the present invention, concentration of the alkylsilane in a solvent range from about 0.05 vol % to about 100 vol %.

In an embodiment of the present invention, the solvent is selected form N,N-dimethylformamide (DMF), N,N-diethylformamide (DEF), methanol, ethanol, ethyl ether, dichloromethane, tetrahydrofuran (THF), toluene, pyridine, hexane, benzene or combination thereof.

In an embodiment of the present invention, the elevated temperature is in a range of 60° C. and 190° C., preferably in a range of 70° C. and 140° C.

In an embodiment of the present invention, the three-dimensional melamine sponge is dried at a temperature in a range of about 100-110° C. for about 0.25-12 hours.

According to one another aspect of the present invention, a method or process for manufacturing a modified porous composite structure includes steps as follows:
The porous structure such as in foams or sponges may be open cell foam, ultra-light density (6-12 kg/m³). Alternatively, the structure may be woven or non-woven material.
In an embodiment, the substrate is melamine foam, or any three-dimensional sponge/structures known in the art.
The first step modification involves contacting the three-dimension structure with pre-grown bacterial cultures in range of 25-40° C. for 24-72 hours. In certain embodiments, the bacterial cultures may include, but not limited to, at least one of Acidobacteriota, Acidithiobacillales, Thiobacillus, Acetobacter, Alicyclobacillus etc or a combination thereof in equal ratio. Bacterial cultures that can proliferate rapidly and produce biological acids and enzymes during their metabolic process are considered to exploit biological etching of surfaces. After treatment surface roughness with heights fluctuating up to 130 μm is achieved.
The cultures are grown in a mixture of KH₂PO₄ 3.00 g, MgSO₄×7 H₂O 0.50 g, (NH₄)₂SO₄ 3.00 g, CaCl₂×2 H₂O 0.25 g, Na₂S₂O₃×5 H₂O 5.00 g, and Distilled water 1000.00 ml. The medium is autoclaved at 121° C. for 15 minutes ad filter sterilized thiosulfate is added afterwards. Depending on Genus in certain embodiments, thiosulfate is replaced with 5.00 g/l tetrathionate (K₂S₄O₆) sterilized by filtration or 10.00 mg/l FeSO₄×7 H₂O or 10.00 g/l elemental sulfur as substrate or any relevant component required. The final pH will also vary according to bacterial cultures used for etching. The cultures were grown 72 hours, in range of 25-40° C., with CFU>10⁸ cells/ml before inoculating.
Second step modification was coated with any two-dimensional or one-dimensional or transition metal complexes material like reduced graphene, CNTs, or MoS₂@X (X=CNTs or GO) or g-C3N4 (hydrophobic).
The concentration of the two-dimensional or one-dimensional or transition metal complexes in the solvent may range from about 1 ppm to 5000 ppm.
The process for preparing hydrophobic graphitic carbon nitride (g-C3N4) involves: Initially, 10 grams of melamine precursor was heated in a tube furnace at 550° C. for 3 hours under an inert atmosphere to synthesize g-C3N4. Subsequently, the resulting g-C3N4 powder was milled for 30 minutes at a rotation speed of 500 rpm, achieving a particle size below 100 nanometers. Next, 5 grams of the purified g-C3N4 powder were dispersed in 200 mL of toluene solvent, followed by the addition of 0.5 grams of hexamethyldisilazane (HMDS) silane coupling agent. The dispersion was stirred at 800 rpm for 4 hours at room temperature and sonicated for 30 minutes to promote the reaction between the g-C3N4 surface and the silane coupling agent. Subsequently, the modified g-C3N4 dispersion was centrifuged at 4000 rpm for 20 minutes to separate the powder from the solvent. Finally, the hydrophobic g-C3N4 powder was dried in a vacuum oven at 60° C. for 24 hours to remove any residual solvent. The hydrophobicity of the synthesized material was confirmed by measuring the contact angle, which exceeded 152° C.
The process for preparing reduced graphene oxide involves: 1.5 g of the graphite oxide was dispersed in about 1500 mL of water and ultrasonicated for 30 minutes then, maintaining at 90° C. After 1 h, few drops of ammonia solution were added to maintain the pH value at 11. Then followed by the addition of 1 mL hydrazine monohydrate, the solution was stirred for yet another 3 hours. The solution was dried in oven at 80° C. to yield the rGO powder.
Synthesis of MoS₂@CNTs and MoS₂@GO: In a typical reaction, 100 mg of CNTs were dispersed in 30 ml of deionised (DI) water and sonicated for 1 hr to break up any agglomerates and ensure uniform distribution of the CNTs. Separately, 100 mg of MoS₂ was dispersed in 10 ml of DI water, and added drop wise to the CNTs dispersed CNTs while stirring. Further, the mixture was transferred in stainless Teflon apparatus and kept in oven at 90° C. for 12 h. The MoS₂@GO was then filtered off and dried it. Similar, MoS₂@GO composites were prepared using the same ratio of MoS₂ and GO as MoS₂ and CNT, respectively.
The third step modification of the above three-dimension etched structure may be achieved by contacting the structure with an alkylsiloxanes. In certain embodiments, the alkylsiloxane may include at least one of Polyvinyl siloxane, polyethermethylsiloxanes (PEMS), hexamethyldisiloxane, hexamethylcyclotrisiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, Polydimethylsiloxane or a combination thereof.
The concentration of the alkylsiloxane in the solvent may range from about 0.05 wt. % to about 100 wt. %. The structure may be contacted with the alkylsilane for about 10 seconds, to about 300 minutes or more. The solvent can be, but is not limited to, N,N-dimethylformamide (DMF), N,N-diethylformamide (DEF), methanol, ethanol, ethyl ether, dichloromethane, tetrahydrofuran (THF), toluene, pyridine, hexane or benzene. The aforementioned solvent can be used alone or in combination thereof
The dipped structure may be cured at elevated temperatures. In certain embodiments, the temperature may be between 60° C. and 190° C., such as between 70° C. and 140° C. In other embodiments, the drying temperature is about 110° C. The three-dimensional structure may be dried until all the solvent has been removed, for example between about 0.25-12 hour.
The fourth step modification of the above modified layered three-dimensional structures may be achieved by contacting the structure with an alkylsilane to enhance both hydrophobicity and hierarchical micro-nano bulges due to its long chain side groups. The alkyl group of the alkylsilane may be aromatic, straight or branched chain aliphatic, or a fluorohydrocarbon. The alkyl group may be substituted and may be unsaturated at one or more bonds. In certain embodiments the alkylsiloxanes include alkyl groups of hydrocarbons or fluorohydrocarbons of about 1 to about 30 carbon atoms. In certain embodiments, the alkylsilane may include at least one of octadecyltrichlorosilane, dodecyltrichlorosilane, octyltrichlorosilane, butyltrichlorosilane, Hexyltrichlorosilane, or a combination thereof.
The concentration of the alkylsilane in the solvent may range from about 0.05 vol % to about 100 vol %. The substrate may be contacted with the alkylsilane for about 10 seconds, to about 300 minutes or more. The solvent can be, but is not limited to, N,N-dimethylformamide (DMF), N,N-diethylformamide (DEF), methanol, ethanol, ethyl ether, dichloromethane, tetrahydrofuran (THF), toluene, pyridine, hexane or benzene. The aforementioned solvent can be used alone or in combination thereof.
Unreacted alkylsiloxane and alkylsilane may be removed from the modified structure by pressing or squeezing the absorbed solution. The modified structure may be repeatedly washed with solvent.
The washed structure may be cured at elevated temperatures. In certain embodiments, the temperature may be between 60° C. and 210° C., such as between 70° C. and 140° C. In other embodiments, the drying temperature is about 110° C. The three-dimensional structure may be dried until all the solvent has been removed, for example between about 0.25-12 hour.

The present invention results in triple roughened surface achieved by facile Melamine foam/3D matrix exfoliation at room temperature and environmentally safe conditions using different acidophiles, any two-dimensional or one-dimensional or transition metal complexes material coated on bacterial etched surface, PDMS coating as anchoring layer on bacterially etched foam resulting in higher WCAs and low contact angle hysteresis, followed by OTS coating for multiple cycles of sorption-squeezing process where coatings are not detached on squeezing and there is insignificant change in oil sorption capacity of modified material.

### EXAMPLES

### Example 1: Preparation of Hydrophobic Three-Dimensional Sponge

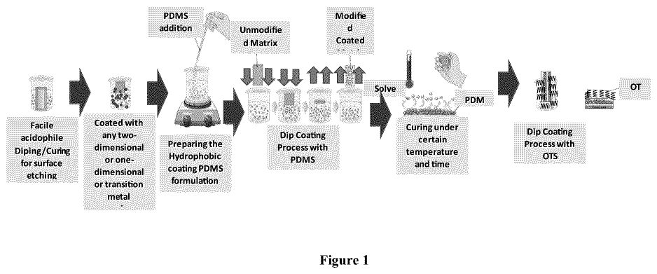

a) Melamine sponge (oleophilic with a water contact angle≈0°) is purchased from M/s Supremex Industries, India and used as received. Other chemicals were purchased from Sigma (India). To modify this amphiphilic structure to superhydrophobic-superoleophillic, it was first cut into small pieces (4×4 cm), and then immersed in 72 hours pre-grown bacterial solution for 24 hours at room temperature. After this it was washed with distilled water twice and dried at 80° C. The 3D structure was dipped into a suspension of reduced graphene oxide (reduced using hydrazine) and autoclaved at 120° C. for 2 hours. It offers mechanical flexibility and remains adhered due to strong van der Waals interactions. Thereafter, it was dipped in 1.25% polydimethylsiloxane (PDMS) in toluene for 2 hours at 65° C. The absorbed solution was squeezed out it was dried in an oven at 110° C., for cementing the reduced graphene sheets on the sponge. Fourthly, it was facile dip in 100 Trichloro(octadecyl)silane (OTS) in toluene for 2.5 hours at 70° C. The absorbed solution was squeezed out it was dried in an oven at 120° C. This converts hydrophillic three-dimensional melamine foam to hydrophobic in nature (FIG. 1). Even after cutting into several smaller pieces, it illustrated hydrophobic character. The sequence of modification process is important for development of foam with desired phenotypes as elucidated in table below, where:

| | Water | |
|---|---|---|
| | Contact | |
| | Angle | Absorption |
| Treatment sequence | (WCA) | (g/g) |
| Facile acidophile incubation → Reduced Graphene sheet | 158° | 113 |
| → PDMS→ OTS | | |
| Reduced Graphene sheet → PDMS→ OTS → Facile | 93° | 68 |
| acidophile incubation | | |
| PDMS→ Facile acidophile incubation → Reduced Graphene | 87° | 57 |
| sheet → OTS | | |
| Facile acidophile incubation → Reduced Graphene sheet → | 118° | 73 |
| OTS →PDMS | | |
| OTS → PDMS→ Reduced Graphene sheet → Facile | 107° | 84 |
| acidophile incubation | | |
| Facile acidophile incubation → OTS →PDMS→ Reduced | 128° | 99 |
| Graphene sheet | | |

b.) Melamine sponge (oleophilic with a water contact angle −0°) is purchased from MVJs Supremex Industries, India and used as received. Other chemicals were purchased from Sigma (India). To modify this amphiphilic structure to superhydrophobic-superoleophillic, it was first cut into small pieces (4×4 cm), and then immersed in 72 hours pre-grown bacterial solution for 24 hours at room temperature. After this it was washed with distilled water twice and dried at 80° C. Second step involves, dipping in MOS₂@CNTs. Thereafter, it was dipped in 1.25% PDMS in toluene for 2 hours at 65′° C. The absorbed solution was squeezed out it was dried in an oven at 110° C., for cementing the reduced graphene sheets on the sponge. Fourthly, it was facile dip in 100 OTS in toluene for 2.5 hours at 70° C. The absorbed solution was squeezed out it was dried in an oven at 120° C. This converts hydrophillic three-dimensional melamine foam to hydrophobic in nature. Even after cutting into several smaller pieces, it illustrated hydrophobic character. The sequence of modification process is important for development of foam with desired phenotypes as elucidated in table below, where:

| | Water | |
|---|---|---|
| | Contact | |
| | Angle | Absorption |
| Treatment sequence | (WCA) | (g/g) |
| Facile acidophile incubation → MoS₂@CNTs → PDMS→ | 156° | 111 |
| OTS | | |
| MoS₂@CNTs → PDMS→ OTS → Facile acidophile | 90° | 65 |
| incubation | | |
| PDMS→ Facile acidophile incubation → MoS₂@CNTs → | 84° | 52 |
| OTS | | |
| Facile acidophile incubation → MoS₂@CNTs → OTS | 111° | 70 |
| →PDMS | | |
| OTS → PDMS→ MoS₂@CNTs → Facile acidophile | 101° | 81 |
| incubation | | |
| Facile acidophile incubation → OTS →PDMS→ | 126° | 90 |
| MoS₂@CNTs | | |

Thus, both the above examples illustrated that the sequence of modification process is important for development of foam with desired phenotypes, i.e., WCA and absorption efficiency.

Example 2: Effect of acidophile inoculation for enhanced roughness and time. Experiment was carried at ambient temperature and pressure, with 72 hours pre-grown bacterial cultures. This was followed by PDMS and OTS coating as in Example 1.

| | Roughness | Water | |
|---|---|---|---|
| | heights | Contact Angle | Absorption |
| Treatment | (μm) | (WCA) | (g/g) |
| Nil | 10 | 140 | 98 |
| Thiobacillus acidiphillus | 125 | 158° | 111 |
| Thiobacillus ferrooxidans | 130 | 147° | 113 |

The above example shows that the WCA and absorption efficiency improved with acidophile treatment.

Example 3: Effect of PDMS concentration is subsequently investigated for enhanced hydrophobicity. PDMS concentration was varied as tabulated. This was preceded by bacterial etching and followed by OTS coating. The hydrophobic transition of the melamine sponge is not limited to single alkylsiloxane. The PDMS loading on melamine foam was characterized by gravimetric measurements using an analytical balance with an accuracy of 0.001 mg. The absorption efficiency was measured using hexane.

| Treatment with | | | | |
|---|---|---|---|---|
| different | Bulk | | Water | |
| conc. of | density | Porosity | Contact | Absorption |
| PDMS (w/v) | (mg/cm3) | (%) | Angle (WCA) | (g/g) |
| Nil | 8.11 | 99.54 | 110° | 96 |
| 0.25 | 8.19 | 99.50 | 124° | 96 |
| 0.5 | 8.23 | 99.50 | 137° | 98 |
| 0.75 | 8.28 | 99.50 | 149° | 105 |
| 1 | 8.37 | 99.49 | 154° | 109 |
| 1.25 | 8.39 | 99.49 | 158° | 113 |
| 1.5 | 8.41 | 99.45 | 158° | 113 |
| 1.75 | 8.42 | 99.45 | 158° | 113 |
| 2 | 8.44 | 99.43 | 158° | 113 |

Using this example, it was inferred that PDMS concentration is important for achieving maximum WCA and absorption efficiency.

Example 4: Effect of OTS concentration is subsequently investigated for enhanced hydrophobicity. OTS concentration was varied as tabulated. This was preceded by bacterial etching and PDMS coating. The hydrophobic transition of the melamine sponge is not limited to single alkylsilane. The OTS loading on melamine foam was characterized by gravimetric measurements using an analytical balance with an accuracy of 0.001 mg. The absorption efficiency was measured using hexane.

| Treatment with | | | | |
|---|---|---|---|---|
| different | Bulk | | Water | |
| conc. of | density | Porosity | Contact | Absorption |
| OTS (w/v) | (mg/cm3) | (%) | Angle (WCA) | (g/g) |
| Nil | 8.11 | 99.54 | 103° | 71 |
| 0.25 | 8.21 | 99.49 | 123° | 79 |
| 0.5 | 8.24 | 99.49 | 147° | 82 |
| 0.75 | 8.30 | 99.48 | 152° | 99 |
| 1 | 8.39 | 99.47 | 158° | 113 |
| 1.25 | 8.42 | 99.47 | 158° | 113 |
| 1.5 | 8.44 | 99.43 | 158° | 113 |
| 1.75 | 8.46 | 99.42 | 158° | 113 |
| 2 | 8.46 | 99.40 | 158° | 113 |

Using this example, it was inferred that OTS concentration is important for achieving maximum WCA and absorption efficiency.

Example 5: The stability of the prepared superhydrophobic foam at different pH and temperatures was evaluated as follows. The below was followed by drying in oven (110° C.). It was immersed in the below solutions forcibly. Water contact angles and absorption efficiency using hexane were measured after drying.

| | | Water | |
|---|---|---|---|
| | | Contact | |
| | | Angle | Absorption |
| Treatment | Time | (WCA) | (g/g) |
| Nil | Nil | 158° | 113 |
| Boiling water | 30 min | 152° | 108 |
| Dipping in acid (0.1M HCl) | 30 min | 145° | 95 |
| Dipping in acid (0.1M H₂SO₄) | 30 min | 149° | 98 |
| Dipping in alkali | 30 min | 151° | 102 |
| solution (0.1M NaOH) | | | |

The results demonstrated that the sponge retains its strong hydrophobic properties even after exposure to harsh conditions in highly acidic and alkaline environments, making it suitable for use in a variety of extreme weather conditions.

Example 6: The absorption capacity and recyclability of the modified three dimensional was evaluated by repeated sorption-squeezing processes, by immersing the sponges into oils, waiting until the sponge became saturated with the oils, and then squeezing the sponge.

| | Surface | | |
|---|---|---|---|
| | tension | Sorption capacity | Sorption capacity |
| Oil type | in mN/m | (g/g) | after 100 cycles (%) |
| ATF (Lighter than water-the | 23 | 119.91 | 97.6 |
| layer settles above water) | | | |
| Toluene (Lighter than water- | 28.4 | 125.06 | 98.5 |
| the layer settles above water) | | | |
| THF (Lighter than water-the | 26.4 | 113.6 | 97.1 |
| layer settles above water) | | | |
| DMF (Lighter than water-the | 37.10 | 122.4 | 96.9 |
| layer settles above water) | | | |
| Diesel (Lighter than water-the | 27.88 | 120.8 | 95.01 |
| layer settles above water) | | | |
| TUPT Crude (Lighter than | 36.4 | 137.6 | 84.1 |
| water-the layer settles above | | | |
| water) | | | |
| Siberian light Crude (Lighter | 34.1 | 126.4 | 90.9 |
| than water-the layer settles | | | |
| above water) | | | |
| Vegetable oil (Lighter than | 31-35 | 159.5 | 92.8 |
| water-the layer settles above | | | |
| water) | | | |
| CCl4 (Denser than water-the | 28 | 146.3 | 87.1 |
| layer settles below water) | | | |
| Chloroform (Denser than | 27.50 | 171.8 | 95.6 |
| water-the layer settles below | | | |
| water) | | | |

*Surface tension of water is 72.8 mN/m.

Thus, the developed material exhibited very high sorption capacity, ranging from 95 to 170 times (w/w), depending on the polarity and density of the employed organic solvents and oils. Thus, even after 100 cycles of sorption-squeezing>9000 sorption capacity was still present, for different oil types having different polarities, densities, surface tensions and viscosities (low viscosity organic solvents—such as toluene, THF, DMF, and chloroform, & high viscosity oils—such as diesel, mineral oil, and machine oil.).

Example 7: Emulsification of crude oil in synthetic seawater was done with different dispersants to evaluate their effects. The procedure for preparing emulsions is crude oil and water is mixed in desired ratio, thereafter dispersant is added. The sample is then mixed using a vortex mixer at 3000 rpm for 30 s to create the oil/water (O/W) emulsion.

| | Absorption |
|---|---|
| | capacity |
| Treatment type | (g/g) |
| Emulsified oil (with Tween 80) in synthetic seawater | 98 |
| Emulsified oil (with Tween 20) in synthetic seawater | 105 |
| Emulsified oil (with sodium lauryl sulfate) in synthetic | 95 |
| seawater | |
| Emulsified oil (with sodium dodecyl sulfate) in synthetic | 89 |
| seawater | |
| Immiscible oil in synthetic seawater | 113 |

It confirms the utility of developed material for absorption of emulsified oil in sea water conditions.

Example 8: Effect of incubation time on WCA (i.e., hydrophobicity) of the modified sponge achieved at the end of modification process. The absorption efficiency was measured using hexane.

| | | Water Contact | Absorption capacity |
|---|---|---|---|
| Treatment type | Time in hours | Angle (WCA) | (g/g) |
| Facile acidophile | 12 | 147 | 110 |
| incubation | 24 | 158 | 113 |
| | 36 | 155 | 109 |
| | 48 | 152 | 105 |
| PDMS dip | 1 | 152 | 105 |
| | 2 | 158 | 113 |
| | 3 | 158 | 109 |
| | 4 | 158 | 103 |
| OTS dip | 1.5 | 150 | 107 |
| | 2.5 | 158 | 113 |
| | 3.5 | 158 | 103 |
| | 4.5 | 158 | 99 |

The process for development of oleophillic and hydrophobic material is sequence and time dependent for achieving enhanced performance and this has been clearly demonstrated in above examples.

### Advantages of the Present Invention

The advancements of the present invention over prior arts are:
Melamine foam/3D matrix exfoliation at room temperature and environmentally safe conditions using different acidophiles. The process is moderate and clean in comparison to conventional methods using strong acids.
The above results in enhanced surface roughness by creating hierarchical micro- and nano-roughness to achieve super non-wetting conditions, and here it was directed with facile bacterial method.
D Coating on etched surface with two-dimensional or one-dimensional or transition metal complexes material like reduced graphene, CNTs, or MoS₂@X (X=CNTs or GO) or g-C3N4 (hydrophobic) for enhanced oil sorption capacity.
In third step PDMS was used to coat bacterially etched foam/coated with 1D/2D materials for imparting hydrophobic coating, as it dramatically reduces the surface energy while acting as anchoring layer without resulting in any change in the morphology/porosity of the matrix used.
In PDMS coating having crosslinked Si—O—Si bond the mechanical stability along with flexibility is increased which is required for multiple cycles of sorption-squeezing process, because it can be irreversibly bound to sponge materials without the use of adhesives.
The PDMS coating done over bacterial etched surface results in higher WCAs and low contact angle hysteresis in comparison to direct PDMS coating over the sponge.
The fourth treatment with OTS, results in generation of rougher micro-nano bulges. This is due to low surface energy spiny structures of OTS because of its long chain carbon length. Additionally, OTS polymerizes by self-dehydration (forming a self-assembled monolayer (SAM) and condensation resulting in micro-nano scale level of roughening. This is covalently bonded on the layers of material. In comparison to direct OTS coating over sponge this results in enhanced roughness (i.e. super non-wetting surface), much high WCAs, low contact angle hysteresis, higher absorption efficiencies.
Unlike previous super-hydrophobic sponges in which hydrophobic coatings easily detaches on squeezing, the present invention discloses bonded attachment on the materials, which remains unaffected by the squeezing process and exhibited outstanding recyclability with oil sorption retention even after 1000 cycles of sorption-squeezing, enhanced by bacterial etching followed by PDMS and OTS coating.
The entire process is directed to make it super-hydrophobic synergistically. The benefit of coating OTS on the surface of PDMS present over 1D/2D structures on the microbially etched surface results in superhydrophobicity with remarkable roughness. Thus, low surface energy material in combination with rough structure made the material superhydrophobic for trapping air at the interface to prevent the material from being soaked with water. This triple roughened surface, as inspired by a lotus leaf, provides sufficient superhydrophobic property.
The developed foam shows stability under extreme pH (1-13) and temperatures (0-60-C), with all WCAs are around 150°.
There is no leaching of material resulting in sustained superhydrophobicity.
There is not much changes in porosity (i.e., decline in porosity is <2%).
Due to modification, there is only slight increase in bulk density (<5%).
The developed material can separate oil from water in immiscible as well as emulsified form.
The developed material can separate both high (forming lower layer in oil-water mixture) and low-density oils (forming upper layer in oil-water mixture) from the water.
The process of modification is economical and facile with greener alternatives for surface etching of the sponge for enhanced surface areas.
The modified three-dimensional materials can take up several varieties of oil and solvents up to 161 times its weight and had separation efficiency as high as 99.99%. Basically, any low surface energy liquid can be recovered from water.

We claim:

1. A process for modification of a melamine sponge, the process comprising:
i. contacting a three-dimensional structure of a melamine sponge with a pre-grown bacterial culture at a temperature in a range of 25° C.-40° C. for 24-72 hours to obtain an etched porous substrate;
ii. coating the etched porous substrate with a one-dimensional metal complex, a two-dimensional metal complex, or a transition metal complex to obtain a coated etched porous substrate;
iii. dip contacting the coated etched porous substrate with an alkylsiloxane dispersed in a first solvent for a duration between 10 seconds to 300 minutes and curing at a temperature between 60° C. and 190° C. to obtain alkylsiloxane coated structure;
iv. contacting the alkylsiloxane coated structure with an alkylsilane dispersed in a second solvent for a duration between 10 seconds to 300 minutes to obtain a modified 3D sponge;
v. washing the modified 3D sponge with a solvent and curing at a temperature between 60° C. and 210° C. to obtain the modified melamine sponge.

2. The process as claimed in claim 1, wherein the pre-grown bacterial culture is selected from the group consisting of Acidobacteriota, Acidithiobacillales, Thiobacillus, Acetobacter, Alicyclobacillus, and a combination thereof.

3. The process as claimed in claim 1, wherein the bacterial culture is grown in a medium comprising potassium dihydrogen phosphate (KH₂PO₄) in an amount of 2-5 g, magnesium sulfate heptahydrate (MgSO₄·7H₂O) in an amount of 0.25-2.0 g, ammonium sulfate [(NH₄)₂SO₄] in an amount of 2-5 g, calcium chloride dihydrate (CaCl₂·2H₂O) in an amount of 0.1-1.0 g, and sodium thiosulfate pentahydrate (Na₂S₂O₃·5H₂O) in an amount of 2-8 g.

4. The process as claimed in claim 1, wherein the one-dimensional metal complex, the two-dimensional metal complex, or the transition metal complex is selected from the group consisting of reduced graphene, CNTs, MoS₂@CNTs, MoS₂@GO, and g-C3N4, wherein g-C3N4 is hydrophobic.

5. The process as claimed in claim 1, wherein the one-dimensional metal complex, the two-dimensional metal complex or the transition metal complex is dispersed in a solvent in a concentration ranging from about 1 ppm to 5000 ppm.

6. The process as claimed in claim 1, wherein the alkylsiloxane is selected from the group consisting of polyvinyl siloxane, polyethermethylsiloxanes (PEMS), hexamethyldisiloxane, hexamethylcyclotrisiloxane, octamethyltrisiloxane, octamethylcyclotetrasiloxane, decamethyltetrasiloxane, decamethylcyclopentasiloxane, dodecamethylpentasiloxane, dodecamethylcyclohexasiloxane, tetradecamethylhexasiloxane, polydimethylsiloxane, and a combination thereof.

7. The process as claimed in claim 6, wherein the alkylsiloxane is in a concentration ranging from about 0.05 wt. % to about 100 wt. %.

8. The process as claimed in claim 6, wherein the alkylsiloxane is polydimethylsiloxane (PDMS).

9. The process as claimed in claim 1, wherein the alkylsilane is selected from the group consisting of octadecyltrichlorosilane, dodecyltrichlorosilane, octyltrichlorosilane, butyltrichlorosilane, hexyltrichlorosilane, and a combination thereof.

10. The process as claimed in claim 9, wherein the alkylsilane is in a concentration ranging from about 0.05 vol % to about 100 vol %.

11. The process as claimed in claim 9, wherein the alkylsilane is octadecyltrichlorosilane (OTS).

12. The process as claimed in claim 1, wherein the first solvent is selected from the group consisting of N,N-dimethylformamide (DMF), N,N-diethylformamide (DEF), methanol, ethanol, ethyl ether, dichloromethane, tetrahydrofuran (THF), toluene, pyridine, hexane, benzene, and a combination thereof.

13. The process as claimed in claim 1, wherein the second solvent is selected from the group consisting of N,N-dimethylformamide (DMF), N,N-diethylformamide (DEF), methanol, ethanol, ethyl ether, dichloromethane, tetrahydrofuran (THF), toluene, pyridine, hexane, benzene, and a combination thereof.

14. The process as claimed in claim 1, wherein the curing is carried out at a temperature in a range of 70° C. and 140° C.

15. The process as claimed in claim 1, further comprising drying the modified melamine sponge at a temperature in a range of about 100° C.-110° C. for about 0.25-12 hours.

16. The process as claimed in claim 1, wherein the modified melamine sponge has a water contact angle of about 150°, a sorption capacity in a range of 95 to 170 (g/g).

17. The process as claimed in claim 1, wherein the modified melamine sponge is stable under extreme pH ranging from 1-13 and at a temperature in a range of 0° C.-60° C.

18. The process as claimed in claim 1, wherein the modified melamine sponge has a porosity of about 99.5%.

19. The process as claimed in claim 1, wherein the modified melamine sponge has a bulk density of about 8.4 mg/cm³.
